(19) United States
(12) Patent Application Publication
(10) Pub. No.: US 2018/0339082 A1
(43) Pub. Date: Nov. 29, 2018

(54) POLYETHER ETHER KETONE SURFACE-MODIFIED WITH HYDROXYAPATITE

(71) Applicant: Bioalpha Corporation, Gyeonggi-do (KR)

(72) Inventors: Mi Young Ryu, Gyeonggi-do (KR); Jun Hyuk Seo, Gyeonggi-do (KR); Hyun Seung Ryu, Gyeonggi-do (KR); Nam Yul Cho, Gyeonggi-do (KR); Hak Su Jang, Gyeonggi-do (KR)

(21) Appl. No.: 15/602,903

(22) Filed: May 23, 2017

(51) Int. Cl. A61L 27/12; A61L 27/18

(52) U.S. Cl. CPC A61L 27/12; A61L 2400/18; A61L 2430/02; A61L 27/18

(57) ABSTRACT

The present invention relates to a polyether ether ketone (PEEK)-based material comprising hydroxyapatite (HA) particles which are embedded discontinuously on the surface thereof, a method of preparing the PEEK-based material, and a bone graft comprising the PEEK-based material.

### TECHNICAL FIELD

The present invention relates to a polyether ether ketone (hereinafter, referred to as PEEK)-based material which includes hydroxyapatite (HA) particles embedded discontinuously on the surface thereof, a method of preparing the PEEK-based material, and a bone graft material which includes the PEEK-based material.

### BACKGROUND ART

Polyether ether ketone (hereinafter, referred to as PEEK) is a material widely used as a material for manufacturing bearings, pistons, valves, etc., which must undergo severe abrasion, due to its excellent physicochemical properties such as excellent wear resistance and tensile strength. Since the late 1990s, PEEK has been used as a bone-substituting material after it was confirmed to maintain intrinsic physical properties while not inducing any adverse reactions in vivo.

However, due to the bio-inertness of PEEK that does not react with its neighboring tissue after being grafted into the body, it does not fuse with the neighboring bone tissue but simply serves the role of filling in the region of loss thereby maintaining the mechanical strength of the region. As shown in FIG. 1, when PEEK is grafted, it does not show any adverse reactions in the grafted region but the bone tissue newly formed from the neighboring region is detached therefrom. Meanwhile, hydroxyapatite (hereinafter, referred to as HA) has the same component as that of bone tissue and thus the bone tissue newly formed from the neighboring region after transplantation is well attached to the graft material and formed therein (see FIGS. 1(a) and 1(b), respectively). As such, since the neighboring bone does not attach to the PEEK-containing graft material, the graft material is sometimes easily detached. Additionally, a non-fusion between the bone tissue in the grafted region and PEEK may induce absorption of the existing bone tissue according to the micro-motion of tissue by the movement of the human body and the relating pain may be worsened.

To remedy the above drawbacks of PEEK, various methods have been suggested including a method of strengthening the convexo-concave structure of the surface, or a method of inducing a physical attachment with the neighboring tissue by forming pores, or a method of mixing a material such as HA and carbon nanotubes or coating them on the surface, etc. However, the methods of sophisticating the formation of PEEK surface or increasing the area shared with the newly-formed bone tissue by enlarging the cross-sectional area is not a fundamental resolution to overcome the non-fusion. Accordingly, continued studies on the methods of mixing an osteoconductive material such as HA, etc., with PEEK or coating the same on the PEEK surface are being conducted.

When a graft material is prepared by mixing a ceramic material such as HA with a polymer, it generally results in affecting the intrinsic properties of polymers such as tensile strength and ductility. The same physical change occurs even when HA is mixed along with PEEK, and as a result, it was confirmed that tensile strength and fatigue strength are reduced according to the mixed amount of HA. Accordingly, studies are being focused on various coating methods which can induce a fusion with a neighboring tissue while maintaining high tensile strength, excellent wear resistance, high fatigue strength, etc., which are physical properties of PEEK.

Hydroxyapatite (Ca₁₀(PO₄)₆(OH)₂) has hydroxy (OH⁻) groups and phosphate (PO₄³⁻) groups exposed on its surface and thus exhibits hydrophilicity. Due to the hydrophilicity, hydroxyapatite has an excellent wetting property to water but it has very low reactivity to general polymer materials. PEEK is a representative hydrophobic material having excellent resistance and chemical resistance. Due to such characteristics, it is impossible to form a chemical bond between PEEK and HA.

As described above, the coating of hydrophilic HA on the surface of hydrophobic PEEK cannot be achieved by a general chemical bond and thus a plasma coating method is mainly used. However, when the HA ionized in high-temperature/high-speed conditions is applied to the PEEK surface, there is a problem in that the ratio between calcium and phosphorous contained in the final HA cannot be maintained. Additionally, when PEEK (melting point about 340° C.) is used, the structure of the PEEK graft material may be locally modified due to the plasma temperature of HA (about 1,000° C.) because PEEK exhibits a glass transition temperature in a range of 130° C. to 150° C. Furthermore, PEEK has a disadvantage in that even when the PEEK surface is coated with HA, the entire coated surface may be detached due to the low binding affinity on the interface.

### DISCLOSURE

### Technical Problem

Under the circumstances, the present inventors have made efforts to develop a graft material which has improved osteoconductivity and enables maintaining the tensile strength of PEEK, by modifying the surface of PEEK with high tensile strength using HA particles which have the same component as that of bone tissue. As a result, the present inventors have confirmed that when a PEEK graft material including HA islands, which are formed by HA particles embedded discontinuously on the surface thereof, can have osteoconductivity while being capable of maintaining the high tensile strength and excellent wear resistance of PEEK itself using HA particles in an appropriate size and applying a relatively low discharge pressure to a blasting process, which is a general method for treating PEEK graft materials, and can thus provide a graft material enabling a fusion with a neighboring bone tissue when grafted into the body, thereby completing the present invention.

### Technical Solution

A first aspect of the present invention provides a PEEK-based material including HA particles which are embedded discontinuously on the surface thereof.

A second aspect of the present invention, as a method for preparing the PEEK-based material according to the first aspect, provides a method for preparing a PEEK-based material which includes a first step of preparing a PEEK-based material in a predetermined form; and a second step of performing a blasting process on the PEEK-based material using HA particles.

A third aspect of the present invention provides a bone graft material which includes the PEEK-based material according to the first aspect.

Herein-after, the present invention is explained in detail.

As used herein, the term “polyether ether ketone (PEEK)” refers to a kind of an organic thermoplastic polymer that belongs to the polyaryletherketone (PAEK) family. Since PEEK is a semicrystalline thermoplastic resin which has excellent mechanical and chemical characteristics that can be maintained at high temperatures, it is thus widely used in industries. PEEK has a glass transition temperature in a range of 130° C. to 150° C. and a melting point of about 340° C. It is highly resistant to thermal degradation, as well as to attack by both organic and aqueous environments. Due to high tensile strength, PEEK is used for preparing items, which are used in strength-demanding applications, including bearings, piston parts, pumps, HPLC columns, compressor plate valves, and cable sheath. Additionally, PEEK is one of the few plastic materials suitable for the application of ultra-high vacuum. Furthermore, PEEK is also considered as an improved biomaterial to be used for medical implants. In particular. PEEK has excellent wear resistance and can endure loads and can thus be used as a bone tissue replacement, e.g., a material to replace damaged vertebrae.

As used herein, the term “hydroxyapatite (HA)” refers to calcium apatite in the form of a naturally-occurring mineral having the chemical formula of Ca₅(PO₄)₃(OH) and is normally described as Ca₁₀(PO₄)₆(OH)₂ so as to represent the crystal unit including two entities. HA may be a hydroxyl endmember of a complex apatite group. The OH⁻ ions are substituted with fluoride, chloride, or carbonate to form fluorapatite, chlorapatite, etc. Bone mineral is a modified form of HA. Carbonated calcium-deficient hydroxyapatite is a major component that constitutes dental enamel and dentin. HA may be present in dental and bone tissues of the human body. Accordingly, HA is widely used as a filler to replace cut-off bone tissues or as a coating agent to promote ingrowth of bone tissue into a prosthetic implant.

The present invention is characterized in that with respect to the use of PEEK having high tensile strength as a graft material, the surface of PEEK is modified with HA having a similar component to that of bone tissue in order to overcome the drawback of having a low fusion with bone tissue thereby significantly improving the fusion with bone tissue while being capable of maintaining the high tensile strength of PEEK itself. In particular, the present invention is characterized in that HA particles are embedded discontinuously on the surface of PEEK to form islands using HA particles having a predetermined size, considering that a coated film may be detached when the entire surface of PEEK is coated because it is difficult that hydrophobic PEEK and hydrophilic HA form a firm binding between them due to the difference in their properties.

Preferably, the PEEK-based material of the present invention, while including HA particles on the surface thereof, may be in the form where a part of the HA particles is incorporated into the PEEK-based material and the remaining part is projected to the outside of the PEEK surface.

In particular, the HA particles may have a diameter in the range of 100 μm to 450 μm. When the particle size is smaller than 100 μm, the energy (i.e., force) which acts on the powdered raw material during the blasting process is too little for the HA particles to be embedded into the PEEK surface. In contrast, when the particle size is larger than 450 μm, it may result in the etching on the PEEK surface by the blasting process rather than the HA particles are embedded into the PEEK surface.

The method of preparing the PEEK-based material including the HA particles embedded discontinuously on the surface thereof may include a first step of preparing a PEEK-based material in a predetermined form; and a second step of performing a blasting process on the PEEK-based material using HA particles.

As described above, the HA particles are preferred to have a diameter in the range of 100 μm to 450 μm, but the size is not limited thereto.

As used herein, the term “blasting” encompasses all of abrasive blasting, sand blasting, etc., and the blasting is performed to remove rough parts or excessive surface materials on the surface of the raw material or finishing up the surface by a strong spray of a liquid or gas containing an abrasive material using air pressure, oil pressure, or a centrifugal force. Fine abrasion may be performed depending on the kind of the abrasive material.

Preferably, the blasting process may be achieved by a dry blasting method but is not limited thereto, and the blasting process may be performed using various kinds of methods known in the art that can provide a material in which HA particles having a predetermined size are embedded in the form of a discontinuous island on the form of the PEEK surface, without limitation.

More preferably, the blasting process may be performed at a discharge pressure of 2 bar to 4 bar. This discharge pressure range is relatively low compared to the discharge pressure range used for a general blasting process, i.e., a discharge pressure of 6 bar to 7 bar. When the discharge pressure exceeds 4 bar, a coating of the entire surface or surface polishing may occur according to the size of the particle. For example, when the particle size is smaller than 100 μm, the entire coated layer with low tensile strength may be plated over the entire surface, whereas when the particle size is larger than 450 μm surface, polishing may occur. Meanwhile, when the spraying is performed at a discharge pressure of less than 2 bar, a sufficient force cannot be delivered to the HA particles being sprayed and thus the HA particles may not be sufficiently embedded into the PEEK-based material and subsequently removed during the ultrasonication treatment. Even when the HA particles remain, they may soon be detached without being retained for a long period of time thus not being able to achieve the purpose of the present invention to improve the bone fusion of a PEEK-based material.

The present invention is characterized in that HA islands are formed by artificially embedding HA particles into the PEEK surface using a physical force. That is, an object of the present invention is to provide a PEEK-based material which includes on the PEEK surface thereof the HA islands, which are firmly formed not to be detached by a movement or subsequent abrasion occurring therefrom with the neighboring tissue when inserted into the body by removing all of the surface particles that are loosely bound to or embedded into the surface (i.e., removable by applying a predetermined amount of a physical force). The removable HA particles may be those HA particles which remain on the surface because the degree of embedment is low or due to a simple electrostatic attraction. Accordingly, for the removal of the HA particles which are loosely attached to the surface and can be detached later, a step of simple stirring in an aqueous solution, high-speed spraying of an aqueous solution, or ultrasonication treatment of an aqueous solution may additionally be performed after the second step. However, the method of removing the detachable HA particles is not limited thereto, but any method known in the art which can selectively detach the HA particles that are loosely attached to the surface by a force lower than the predetermined binding force may be used without limitation.

Accordingly, more preferably, the washing step may be performed by ultrasonication treatment after the second step. Additionally, the ultrasonication treatment may be performed for 10 minutes to 60 minutes, but is not limited thereto. For example, the method and time of treatment are not limited to those described above as long as the purpose of removing the HA particles loosely attached to the surface can be achieved, and in the case of ultrasonication treatment, it is obvious that the time for treatment may be adjusted according to the intensity of the ultrasonication.

The PEEK-based material according to the present invention which includes the HA particles embedded discontinuously on the surface thereof may be used as a bone graft material.

As used herein, the term “bone graft material or bone implant material”, also called osteograft material, refers to a material which can replace bone tissue and/or a material which can promote new osteogenesis when grafted into damaged or lost bone tissue. Like other tissues in the body, bone tissue also has the ability of self-regeneration but it requires time until the damaged tissue returns to normal and it may be often impossible to achieve complete recovery by self-regeneration alone. Additionally, considering the role of bone tissue as a skeletal frame supporting the human body, the damage in the bone tissue may cause significant inconveniences in terms of supporting and behaviors of the body thus requiring rapid recovery. Therefore, the use of bone graft material may be considered for the above purpose.

In order to fulfill the roles of supporting the body and performing behaviors, the bone graft material is preferred to have not only high tensile strength to endure the body weight of the body and excellent wear resistance to prevent easy wear-out by abrasion with the neighboring tissues such as bone tissue, etc., during the movement of the body, but also the ability to be well-fused with its neighboring tissues to avoid being dislocated from its original position or pressurizing on its neighboring tissues.

Preferably, the bone graft material may be used for treating bone disease due to the loss of a spinal cage or bone tissue, but is not limited thereto.

As used herein, the term “spinal cage” refers to a medical device which is used for a fusion among surgical treatments of degenerative spinal disease. A spinal cage has the roles of securing a space for supplying a bone to be inserted for the fusion after removing a degenerative disc, elevating the height of the intervertebral body to alleviate the pain, recovering the curvature of the spine, and structurally supporting the vertebral body to recover the biomechanical stability of the spine. The spinal cage may be widely used for the treatment of spinal disease such as spinal stenosis, lumbar herniated intervertebral disc, facet joint hypertrophy, etc., which are on the increase along with the aging society, and is being developed by various designs, surgical methods, and materials according to the regions and purposes of use. For example, the spinal cage may be classified into a vertebral body replacement (VBR) for the surgery of removing an intervertebral body and an intervertebral replacement for the fusion of the intervertebral bodies. Additionally, the spinal cage may largely be classified into one for cervical vertebrae and the other for lumbar vertebrae according to the regions for use.

In a specific embodiment of the present invention, each of a PEEK graft material including HA islands, compared to a pure PEEK graft material, was grafted into the ilium of a rabbit, and 8 weeks thereafter, the tensile strength of the grafted graft material was measured while removing it by a simple pulling. As a result, it was confirmed that the PEEK graft material including HA islands showed a 10-fold higher tensile strength than the pure PEEK graft material. This suggests that the neighboring tissue was attached to the PEEK graft material including HA islands. That is, the PEEK-based material, which includes HA particles embedded discontinuously on the surface thereof, according to the present invention, showed a significantly improved ability to fuse with bone while still being able to maintain high tensile strength thus suggesting that the PEEK-based material can be effectively used as a bone graft material.

### Advantageous Effects of the Invention

The PEEK-based material, which includes HA particles embedded discontinuously on the surface thereof, according to the present invention shows a significantly improved ability to fuse with bone while still being able to maintain its intrinsic high tensile strength thus suggesting that the PEEK-based material can be effectively used as a bone graft material.

### BRIEF DESCRIPTION OF DRAWINGS

FIG. 1 shows images of stained bone tissue adjacent to a graft in vivo. FIGS. 1(a) and 1(b) respectively show the results of grafts of PEEK and HA materials.

FIG. 2 shows an image of the surface of a PEEK graft which underwent a final processing. For example, FIG. 2 shows the PEEK surface which underwent a treatment according to the general processing method where silicate powders are sprays at a discharge pressure of 6 bar.

FIG. 3 shows an image illustrating the surface of a PEEK graft which underwent blasting treatment using HA particles with a diameter of at least 300 μm.

FIG. 4 shows an image illustrating the surface of a PEEK graft which underwent blasting treatment using HA particles with a diameter of less than 300 μm.

FIG. 5 shows an image illustrating the surface of a PEEK graft which underwent HA blasting treatment at a discharge pressure of 4 bar.

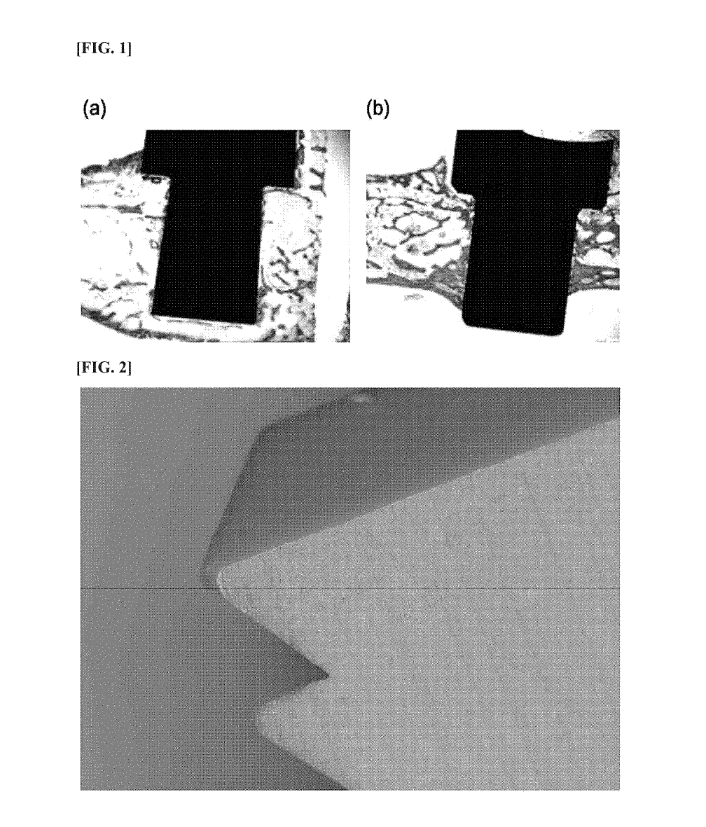

### DETAILED DESCRIPTION OF THE INVENTION

Hereinafter, the present invention will be described in more detail with reference to the following Examples. However, these Examples are for illustrative purposes only and the invention is not intended to be limited by these Examples.

### Example 1: Preparation of a PEEK Graft Material Whose Surface is Treated by HA Blasting

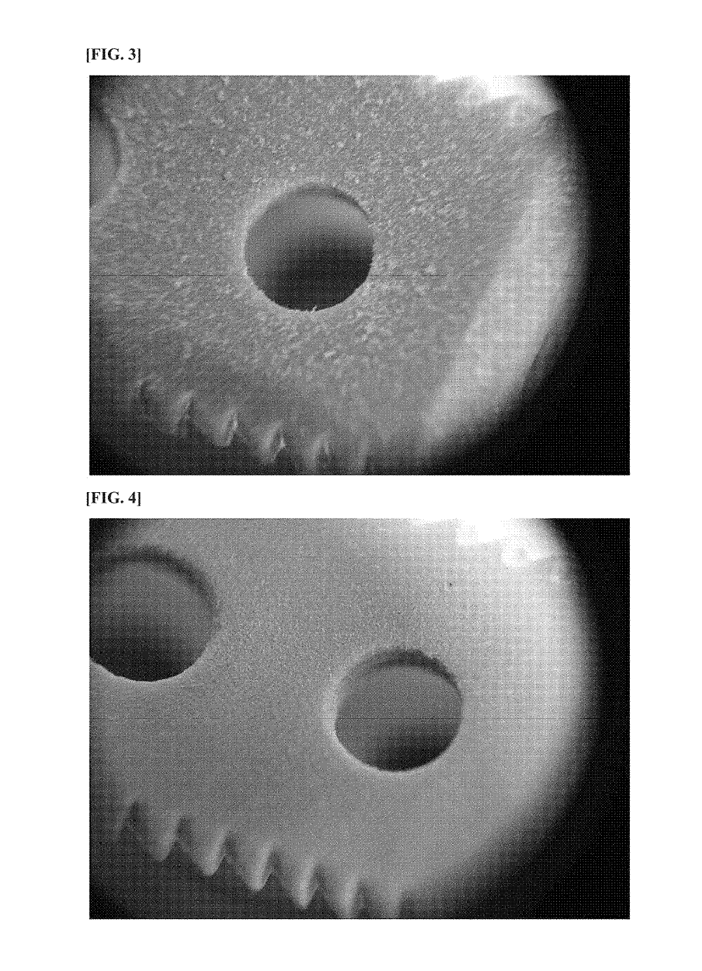

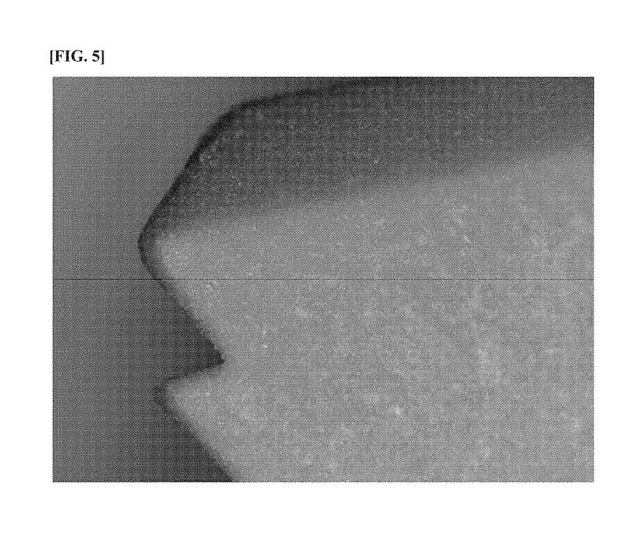

A PEEK graft material, whose processing into a desired form was completed, was fixed on a blasting machine and subjected to a blasting using HA particles with a diameter in the range of 100 μm to 450 μm at a discharge pressure of 4 bar. Then, the PEEK graft material was washed by ultrasonication for 30 minutes and all of the loosely-bound detachable HA particles were removed. The surface images of the PEEK graft materials, which were not treated with HA or blasting-treated, are shown in FIGS. 2 to 5. As shown in FIG. 2, the surface of the PEEK graft material which was not subjected to HA blasting maintained a smooth surface. In contrast, in the case of the PEEK graft materials which were treated with blasting using HA particles with a diameter of at least 300 μm or less than 300 μm, the HA particles were embedded into the surface of the PEEK graft materials and thereby gigantic or micro-islands were formed, as shown in FIGS. 3 and 4.

Meanwhile, when the blasting was performed using HA particles, for confirming the pressure by which the HA particles are sprayed, i.e., for confirming the effect according to the discharge pressure, the blasting was performed not only at the pressure of 6 to 7 bar (i.e., the discharge pressure generally used for blasting) but also at the pressure of 4 bar, which is lower than that and the resulting surface shapes were compared. When the discharge pressure generally used for blasting (i.e., a pressure of 6 bar to 7 bar) was applied, the structures of HA islands according to the present invention which were formed by HA particles embedded discontinuously on the PEEK surfaces were not formed, and there occurred additional abrasive action. In contrast, when the blasting was performed at a pressure of 4 bar, it was confirmed that HA islands were scarcely formed on the PEEK surfaces as shown in FIG. 5. It was confirmed that the thus-formed HA islands were not only more minute but also presented a mild rather than severe convexo-concave structure of the surface compared to those of the existing islands.

It was also confirmed that the surface of the graft material, in which HA islands were formed on the PEEK, which was formed by blasting using HA particles at a discharge pressure lower than that of the general blasting condition, has osteoconductivity and thus enables a fusion with bones. A pure PEEK graft material and the PEEK graft material including the HA islands according to the present invention were grafted into the region of bone loss and the tensile strength with regard to the attachment with the neighboring bone tissue was measured for evaluation. Specifically, the pure PEEK graft material and the PEEK graft material including the HA islands according to the present invention were grafted into the ilium of a rabbit, and 8 weeks thereafter, the tensile strength of the grafted graft material was measured while removing it by a simple pulling. That is, the difference in tensile strength according to the presence/absence of the attachment between the neighboring bone tissue and the graft material was measured and the results are shown in Table 1 below.

TABLE 1

| Period of Grafting | PEEK | PEEK including HA Islands |
|---|---|---|
| 8 Weeks | 18.5N | 193.2N |

As shown in Table 1, it was confirmed that the PEEK graft material including the HA islands showed at least a 10-fold increase in tensile strength compared to that of the pure PEEK graft material. That is, the pure PEEK graft material showed low tensile strength due to the absence of the fusion with the neighboring bone tissue, whereas the PEEK graft material including the HA islands showed significantly increased tensile strength by the attachment with the neighboring bone tissue due to the improved osteoconductivity.

These results suggest that the PEEK graft material including the HA islands according to the present invention can maintain high tensile strength and thus can endure loads when inserted into the intervertebral disc space. As a result, the PEEK graft material including the HA islands according to the present invention can be utilized as an intervertebral spacer suitable for general medical devices, can be used for the treatment of degenerative spinal disease and replace the lost bone tissue when grafted into a lesion with defective bone tissue because the PEEK graft material has an improved fusion with the neighboring bone tissue, and can be effectively used for the treatment of bone disease due to bone loss because the PEEK graft material enables a fusion with bone tissue being newly formed in the neighboring regions.

1. A polyether ether ketone (PEEK)-based material comprising a PEEK material and hydroxyapatite (HA) particles, wherein a surface of the PEEK material is blasted with the HA particles and the HA particles are localized and embedded discontinuously on the surface.

2. The PEEK-based material of claim 1, wherein each of the HA particles has a part incorporated into the surface and the remaining part projecting outside the surface.

3. The PEEK-based material of claim 1, wherein the HA particles have a diameter in the range of 100 μm to 450 μm.

4. A method for preparing a PEEK-based material of claim 1, the method comprising:
a first step of preparing a PEEK material in a predetermined form, and
a second step of performing a blasting process on the PEEK material using the HA particles.

5. The method of claim 4, wherein the HA particles have a diameter in the range of 100 μm to 450 μm.

6. The method of claim 4, wherein the blasting process is achieved by a dry blasting method.

7. The method of claim 4, wherein the blasting process is performed at a discharge pressure of 2 bar to 4 bar.

8. The method of claim 4, further comprising simple stirring in an aqueous solution, high-speed spraying of an aqueous solution, or ultrasonication treatment of an aqueous solution, after the second step.

9-12. (canceled)

13. A polyether ether ketone (PEEK)-based material comprising a PEEK material and hydroxyapatite (HA) particles, wherein
the HA particles are localized on a surface of the PEEK material and discontinuously embedded on the surface by a physical force; and
HA islands are formed on the surface, each of the HA islands having a part incorporated into the surface and the remaining part projecting outside the surface.

14. The PEEK-based material of claim 13, wherein the physical force is applied by blasting the surface with the HA particles.

15. The PEEK-based material of claim 13, wherein the HA particles have a diameter in the range of 100 μm to 450 μm.

16. A method for implanting a PEEK-based material of claim 1 into bone tissues, the method comprising inserting the PEEK-based material into a lesion with defective bone tissues or an intervertebral disc space.

17. The method of claim 16, wherein each of the HA particles has a part incorporated into the surface and the remaining part projecting outside the surface.

18. The method of claim 16, wherein the HA particles have a diameter in the range of 100 μm to 450 μm.

19. The method of claim 16, which is used for treating a bone disease caused by loss of a spinal cage or bone tissues.
